(19) United States
(12) Patent Application Publication
(10) Pub. No.: US 2026/0355430 A1
(43) Pub. Date: Nov. 12, 2026

(54) DEVICE AND METHOD FOR DETECTING INSUFFICIENT LIQUID IN A NEBULIZER

(71) Applicants: OSO-AI, Brest (FR); SORBONNE UNIVERSITE, Paris (FR); INSTITUT NATIONAL DE LA SANTE ET DE LA RECHERCHE MEDICALE, Paris (FR); ASSISTANCE PUBLIQUE HOPITAUX DE PARIS, Paris (FR)

(72) Inventors: Thomas Similowski, Paris (FR); Capucine Morelot-Panzini, Paris (FR); Olivier Menut, Brest (FR); Vincent Geffroy, Brest (FR)

(73) Assignees: OSO-AI, Brest (FR); SORBONNE UNIVERSITE, Paris (FR); INSTITUT NATIONAL DE LA SANTE ET DE LA RECHERCHE MEDICALE, Paris (FR); ASSISTANCE PUBLIQUE HOPITAUX DE PARIS, Paris (FR)

(21) Appl. No.: 19/125,861

(22) PCT Filed: Nov. 6, 2023
(86) PCT No.: PCT/EP2023/080840

§ 371 Date: Apr. 30, 2025

(30) Foreign Application Priority Data
Nov. 7, 2022 (FR) FR2211580

(51) Int. Cl. A61M 11/06; G06N 3/09

(52) U.S. Cl. CPC A61M 11/06; G06N 3/09; A61M 2205/3375; A61M 2205/3379; A61M 2205/50

(57) ABSTRACT

The method of detecting an insufficient quantity of liquid in a reservoir (11) of a nebulizer (10), the nebulizer being configured to form an aerosol from the liquid present in the reservoir, so as to deliver an active ingredient, present in the aerosol, to a user, the method including a) detection, by a microphone (2), of a sound produced, at different times, by the formation of the aerosol; b) processing of the sound detected by a processing unit (3), at each instant, so as to extract at least one feature of the sound; and c) as a function of each feature extracted in step b), detection by the processing unit of an insufficient quantity of liquid in the reservoir for administration of the active ingredient.

### BACKGROUND ART

A nebulizer transforms a liquid drug into an aerosol, i.e. a very fine droplet that reaches the respiratory tract. Among commonly used nebulizers, pneumatic nebulizers generate the aerosol by the action of a gas, typically air, under pressure on a liquid containing an active ingredient. Other nebulizers use ultrasound to produce the aerosol.

Whatever the operating mode, the use of a nebulizer enables an active ingredient to be administered to a patient via the respiratory tract, in the form of small droplets that form an aerosol and are inhaled through the mouth and/or nose. Droplet diameter is generally less than 10 μm.

The liquid containing the active ingredient is held in a reservoir. As the nebulizer is used, the quantity of liquid in the reservoir decreases. Some users may not be able to detect when a reservoir is empty, or unable to stop the nebulizer's operation when the reservoir is empty, or to request assistance when the reservoir is empty. It is therefore important not to leave the nebulizer on the patient's face, for reasons of comfort and, in the case of gas-propelled aerosols, to avoid wasting gas

WO2018/104805 and US2012/017894 describe devices for exploiting a difference in sound intensity when a nebulizer changes from full to an empty state. US2012017894 states that the noise is detected in a spectral band below 100 Hz

It is thus desirable to have an effective, discreet and user-friendly device to verify whether the nebulizer reservoir is sufficiently filled. The invention addresses this need.

### SUMMARY OF THE INVENTION

A first object of the invention is a method of detecting an insufficient quantity of liquid in a reservoir of a nebulizer, the nebulizer being configured to form an aerosol from the liquid present in the reservoir, so as to deliver an active ingredient, contained in the liquid, to a user, the method comprising:
a) detecting, by a microphone, a sound produced, at different times, by the formation of the aerosol;
b) processing of the sound detected by a processing unit, at each instant, so as to extract at least one feature of the sound;
c) as a function of each feature extracted in step b), detecting, by the processing unit, an insufficient quantity of liquid in the reservoir for administration of the active ingredient.

Step c) may comprise:
c1) at least one previously stored criterion is taken into account;
c2) comparison of each feature extracted in step b) with the criterion taken into account in sub-step c1).

Treatment may include:
selection of at least one sound frequency band;
determination of a feature in the selected frequency band.

In one embodiment,
the feature is a spectral power;
the criterion is a spectral power threshold, so that the insufficient quantity of liquid is detected when the spectral power in the selected frequency band exceeds the spectral power threshold.

In one embodiment,
the feature is a spectral power variation;
the criterion is a spectral power variation threshold, so that the insufficient quantity of liquid is detected when the spectral power variation, in the selected frequency band and within a predetermined time interval, exceeds the spectral power variation threshold.

In one embodiment,
the processing unit implements a supervised learning artificial intelligence algorithm;
the supervised learning artificial intelligence algorithm is parameterized by a learning phase that takes into account sounds detected in the absence and presence of an anomaly;

A second object of the invention is a device for detecting an insufficient quantity of liquid in a reservoir of a nebulizer, the nebulizer being configured to form an aerosol from the liquid present in the reservoir, the device comprising:
a microphone, configured to record sounds produced by aerosol formation;
a processing unit, programmed to receive the sounds recorded by the microphone, and to implement steps b) and c) of a process according to the first object of the invention

The invention will be better understood on reading the examples of embodiments presented, in the following description, in connection with the figures listed below.

### BRIEF DESCRIPTION OF THE DRAWINGS

FIG. 1 shows an example of a nebulizer connected to a device according to the invention.

FIG. 2A shows a frequency decomposition of sounds produced when using a nebulizer with a reservoir containing a sufficient quantity of liquid.

FIG. 2B shows a frequency decomposition of sounds produced when using a nebulizer with insufficient liquid in the reservoir.

FIG. 3 shows a schematic diagram of the steps involved in implementing a device according to the invention.

### DETAILED DESCRIPTION OF PARTICULAR EMBODIMENTS

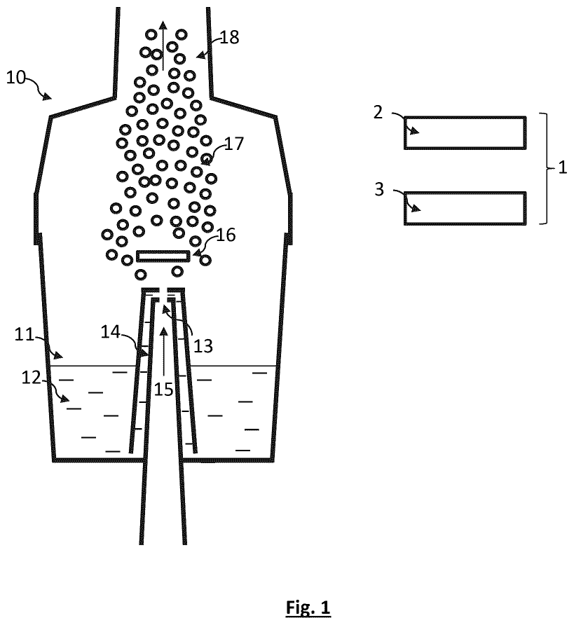

FIG. 1 shows a nebulizer 10 connected to a device 1 according to the invention. The nebulizer 10 comprises a reservoir 11, designed to contain a liquid 12. The nebulizer is configured to form an aerosol from the liquid 12.

In the example shown, the nebulizer is a pneumatic nebulizer, which is a particular example of implementing the invention. The invention applies to other types of nebulizer. The nebulizer comprises an air inlet 13, through which compressed air 15 is admitted to the reservoir. The reservoir 11 has a fluid circuit 14, enabling the liquid in the reservoir to reach the air inlet. The compressed air creates a depression inside the reservoir, enabling the liquid 12 to be pumped through the fluid circuit 14, by a Venturi effect, towards the air inlet 13. At the air inlet 13, the compressed air projects the liquid towards a deflector 16. This results in the formation of an aerosol 17, which propagates towards an outlet 18.

The outlet 18 is connected to an interface designed to be applied against a user's face. The interface may be a respiratory mask or a mouthpiece or a nasal insert.

The device comprises a microphone 2, connected to a processing unit 3. The microphone is configured to record sounds produced by the nebulizer during its operation. More specifically, the microphone is configured to record the noise generated by the nebulization of the liquid inside the reservoir 11.

The processing unit 3 is programmed to process the sounds collected by microphone 2. More specifically, the sounds are produced by the liquid in the reservoir during nebulization.

The inventors observed that when the quantity of liquid in the reservoir becomes low, a characteristic sound signature occurs. This sound signature is attributed to a fluctuation in the quantity of liquid pumped to the air inlet 13. When the quantity of liquid is insufficient, its delivery to the patient is interrupted. It is important to be able to detect this. Thus, analysing the sound recorded by the microphone 2 enables detecting a potential reservoir underfilling.

The processing unit 3 is configured to:
process the sounds recorded by microphone 2 in order to extract features;
compare the extracted features with a criterion, the criterion representing an insufficient reservoir filling;
based on the comparison, determine whether there is an insufficient quantity of liquid in the reservoir.

The criterion representing the insufficient quantity of liquid is determined beforehand during a training phase. It can be:
a spectral power threshold within a predetermined frequency band;
a spectral power variation threshold in a predetermined frequency band, and during a given time period;
a sound intensity threshold or a sound intensity variation threshold.

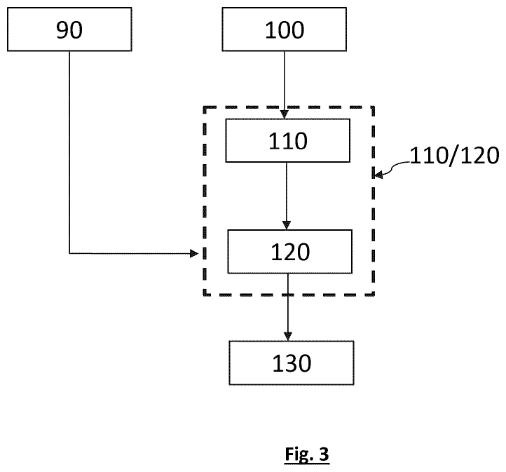

According to one possibility, the processing of the sound resulting from the microphone is carried out by a supervised learning artificial intelligence algorithm, as described below in connection with FIG. 3. The criterion is therefore “implicit”, in the sense that it is embedded in the algorithm's configuration. The output of the algorithm may be the detection or non-detection of a reservoir filling fault.

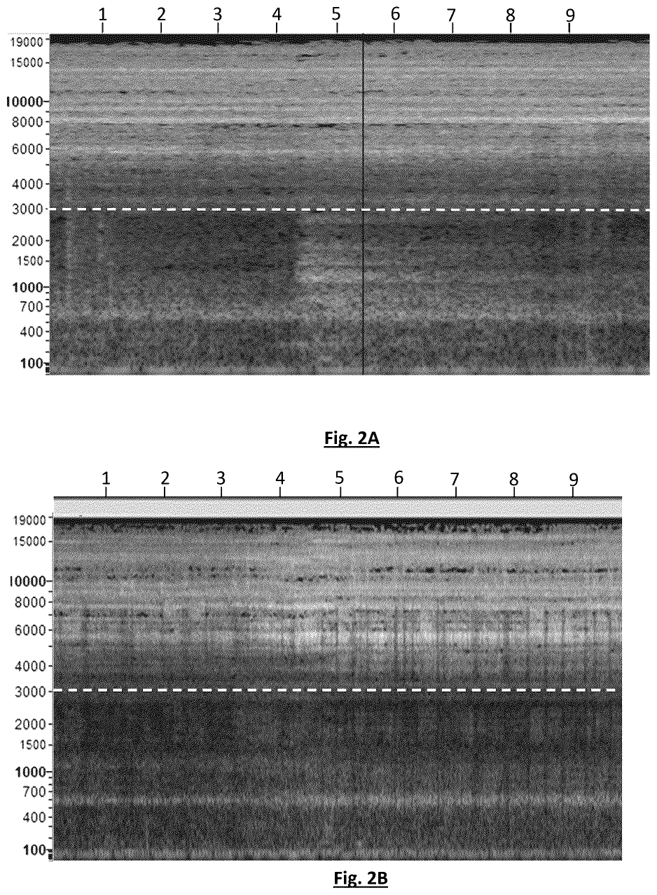

FIG. 2A shows a spectrogram of sounds recorded during a test in which a user used a nebulizer connected to a face mask. During the test, a sufficient quantity of was present in the reservoir.

The x-axis is to time (unit: second). The y-axis is frequency (Hz). The gray level is spectral power.

FIG. 2A shows that in a frequency band between 3000 Hz and 20000 Hz, the spectral power remains relatively stable. This means that the intensity of the acoustic signal from the nebulizer is relatively stable over time in this frequency band.

FIG. 2B shows a spectrogram of sounds recorded during a test similar to the previous one, when the quantity of liquid in the reservoir was insufficient. The axes and gray levels are similar to those in FIG. 2A. It can be seen that in the previously cited 3000 Hz-20000 Hz frequency band, the spectral power undergoes variations, particularly between 3000 Hz and 8000 Hz. In this frequency range, the spectral power varies as a function of time. In FIG. 2B, these variations appear as dark vertical lines. Thus, over a predetermined period of timeΔt, a variation in spectral power, greater than a predefined variation threshold, is detected. The time period Δt is, for example, 1 second. Each spectral power can be measured at a sampling rate of 0.1 s.

The variation threshold, the frequency band and the time period can be adjusted during a training phase. It is understood that the variation threshold and spectral band are to be adjusted according to the type of nebulizer or microphone used.

Thus, in this example, a feature of the sound produced by the nebulizer is extracted at each instant, specifically the variation in spectral power over a time period including said instant. The extracted feature is compared with a criterion, the latter being the previously cited variation threshold. Based on the comparison, an insufficient quantity of liquid in the reservoir is detected or not.

Alternatively, a spectral power threshold criterion can be set for one or more frequencies above 3000 Hz. When, at at least one of these frequencies, the spectral power measured is below the threshold, a filling fault can be detected. The method is more robust if different spectral bands are considered simultaneously.

In FIGS. 2A and 2B, the 3000 Hz threshold has been represented by a dotted line.

FIG. 3 shows the main sound processing stages implemented by the processing unit.
in a step 100, the processing unit 3 receives the sound detected by the microphone;
in a step 110, the processing unit 3 extracts one or more features from the detected sound;
in a step 120, the processing unit 3 takes into account a criterion previously determined in a learning phase 90. The learning phase consists of carrying out tests for one type of nebulizer, with one of sufficient and insufficient filling levels, so as to determine the criterion or criteria corresponding to an insufficient filling level
in a step 130, the processing unit 3 detects an insufficient filling level when at least one or each extracted feature matches the criterion defined in step 120. If an insufficient quantity of liquid is detected, an alarm signal can be generated to alert the user or a team monitoring the user.

In one embodiment, the insufficient filling is detected by implementing a supervised learning artificial intelligence algorithm, such as a neural network. The algorithm is fed either by the sound recorded by the microphone, or by previously extracted sound features.

The output of the algorithm is an indication of whether the filling is sufficient or insufficient. The algorithm is configured during the training phase, taking into account sounds recorded with a sufficient or insufficient quantity of liquid respectively. In this embodiment, the criterion is implicitly considered by the algorithm. Steps 110 and 120 are merged into a single step 110/120, which corresponds to the implementation of the algorithm.

The invention can be used for monitoring nebulizer users, both in a medical environment and at home. It does not require bulky or expensive equipment, making it particularly suitable for home use. Another advantage is that the invention does not require specific nebulizer instrumentation, such as a liquid level sensor in the reservoir. The invention is applicable to various types of nebulizer, beyond the pneumatic nebulizer described in connection with FIG. 1. Preferably, the invention involves a training phase for each type of nebulizer, so as to fine-tune the sound features to extract as well as the criteria to consider.

1. A method for detecting an insufficient quantity of a liquid in a reservoir of a nebulizer, the nebulizer being configured to form an aerosol from the liquid present in the reservoir, so as to deliver an active ingredient, contained in the liquid, to a user, the method comprising:
a) detecting, by a microphone, a sound produced, at different times, by a formation of the aerosol;
b) processing the detected sound, by a processing unit, so as to extract at least one sound feature;
c) based on each sound feature extracted in the processing b), detecting, by the processing unit, an insufficient quantity of the liquid in the reservoir for delivery of the active ingredient;
wherein, in the processing b), the sound feature is extracted within a frequency band in a range of from 3000 Hz to 20000 Hz.

2. The method according to claim 1, wherein in the processing b), the sound feature is extracted within a frequency band in a range of from 3000 Hz to 8000 Hz.

3. The method according to claim 1, wherein the detecting comprises:
c1) taking into account at least one previously stored criterion;
c2) comparing each feature extracted in the processing with the previously stored criterion taken into account in the taking into account c1).

4. The method according to claim 1, wherein the processing b) comprises:
selecting at least one sound frequency band;
determining a feature within the selected frequency band.

5. The method according to claim 4, wherein:
the feature is a spectral power;
the criterion is a spectral power threshold, so that the insufficient quantity of liquid is detected when the spectral power in the selected frequency band crosses the spectral power threshold.

6. The method according to claim 5, wherein:
the feature is a variation in spectral power;
the criterion is a spectral power variation threshold, so that the insufficient quantity of liquid is detected when the variation in spectral power, in the selected frequency band and within a predetermined time interval, crosses the spectral power variation threshold.

7. The method according to claim 1, wherein:
the processing unit implements a supervised learning artificial intelligence algorithm;
the supervised learning artificial intelligence algorithm is configured through a training phase taking into account sounds detected with or without an insufficient quantity of liquid in the reservoir.

8. A device for detecting an insufficient quantity of liquid in a reservoir of a nebulizer, the nebulizer being configured to form an aerosol from the liquid present in the reservoir, the device comprising:
a microphone, configured to record sounds produced by formation of the aerosol;
a processing unit, programmed to receive the sounds recorded by the microphone, and to implement:
b) processing the recorded sounds, so as to extract at least one sound feature;
c) based on each sound feature extracted in the processing b), detecting an insufficient quantity of the liquid in the reservoir for delivery of the active ingredient;
wherein in the processing b), the sound feature is extracted within a frequency band in a range of from 3000 Hz to 20000 Hz.

9. The method according to claim 2, wherein the detecting comprises:
c1) taking into account at least one previously stored criterion;
c2) comparing each feature extracted in the processing with the previously stored criterion taken into account in the taking into account c1).

10. The method according to claim 2, wherein the processing b) comprises:
selecting at least one sound frequency band;
determining a feature within the selected frequency band.

11. The method according to claim 10, wherein:
the feature is a spectral power;
the criterion is a spectral power threshold, so that the insufficient quantity of liquid is detected when the spectral power in the selected frequency band crosses the spectral power threshold.

12. The method according to claim 11, wherein:
the feature is a variation in spectral power;
the criterion is a spectral power variation threshold, so that the insufficient quantity of liquid is detected when the variation in spectral power, in the selected frequency band and within a predetermined time interval, crosses the spectral power variation threshold.

13. The method according to claim 3, wherein the processing b) comprises:
selecting at least one sound frequency band;
determining a feature in the selected frequency band.

14. The method according to claim 13, wherein:
the feature is a spectral power;
the criterion is a spectral power threshold, so that the insufficient quantity of liquid is detected when the spectral power in the selected frequency band crosses the spectral power threshold.

15. The method according to claim 14, wherein:
the feature is a variation in spectral power;
the criterion is a spectral power variation threshold, so that the insufficient quantity of liquid is detected when the variation in spectral power, in the selected frequency band and within a predetermined time interval, crosses the spectral power variation threshold.

16. The method according to claim 2, wherein:
the processing unit implements a supervised learning artificial intelligence algorithm;
the supervised learning artificial intelligence algorithm is configured through a training phase taking into account sounds detected with or without an insufficient quantity of liquid in the reservoir.

17. The method according to claim 3, wherein:
the processing unit implements a supervised learning artificial intelligence algorithm;
the supervised learning artificial intelligence algorithm is configured through a training phase taking into account sounds detected with or without an insufficient quantity of liquid in the reservoir.

18. The method according to claim 4, wherein:
the processing unit implements a supervised learning artificial intelligence algorithm;
the supervised learning artificial intelligence algorithm is configured through a training phase taking into account sounds detected with or without an insufficient quantity of liquid in the reservoir.

19. The method according to claim 5, wherein:
the processing unit implements a supervised learning artificial intelligence algorithm;
the supervised learning artificial intelligence algorithm is configured through a training phase taking into account sounds detected with or without an insufficient quantity of liquid in the reservoir.

20. The method according to claim 6, wherein:
the processing unit implements a supervised learning artificial intelligence algorithm;
the supervised learning artificial intelligence algorithm is configured through a training phase taking into account sounds detected with or without an insufficient quantity of liquid in the reservoir.
